(19) United States
(12) Patent Application Publication
(10) Pub. No.: US 2026/0235537 A1
(43) Pub. Date: Aug. 13, 2026

(54) MEASUREMENT SYSTEM FOR AND METHOD OF MEASURING MOVING WEB

(71) Applicant: Valmet Automation Oy, Espoo (FI)

(72) Inventors: Markku MÄNTYLÄ, Espoo (FI); Pekka SUOPAJÄRVI, Espoo (FI); Janne PAASO, Espoo (FI); Mikko HEIKKILÄ, Espoo (FI)

(73) Assignee: Valmet Automation Oy, Espoo (FI)

(21) Appl. No.: 19/533,695

(22) Filed: Feb. 9, 2026

(30) Foreign Application Priority Data
Feb. 11, 2025 (FI) 20255113

(51) Int. Cl. G01N 23/16; G01N 21/89

(52) U.S. Cl. CPC G01N 23/16; G01N 21/8901; G01N 2223/045; G01N 2223/1016; G01N 2223/304

(57) ABSTRACT

A measurement system for measuring a moving web comprises an X-ray sensor and an optical sensor. The X-ray sensor performs repeatedly electromagnetic transmission measurements of the web. The optical sensor performs optical absorption measurements of the web within an optical range, which includes characteristic optical absorption bands of desired material(s) of the web and at least one reference band. The optical sensor detects optical bands distributed over the optical range independent of both the characteristic absorption bands and the at least one reference band. A number of the optical bands is equal to or larger than a combined number of the characteristic optical absorption bands and the at least one reference band. The optical sensor thus distinguishes the characteristic optical absorption bands and the at least one reference band from each other. At least one of the X-ray sensor and the optical sensor traverse across the moving web. A data processing unit receives data on the measurements, fuses data from the at least two sensors together, and forms information on at least one of the following based on said fused data on absorption: basis weight of the web and a weight per unit area of at least one of the at least one desired material component of the web.

### FIELD

The invention relates to a measurement system for measuring a moving web and a method of measuring moving web.

### BACKGROUND

Measurements of basis weight BW and mass per unit area of different components of a web are important during the manufacturing of paper, board and/or non-woven fabric, for example. The measurements are typically based on NIR (near infrared) radiation and beta-ray absorption. As an alternative to the beta-ray absorption measurement, an X-ray measurement can be performed which is taught in the patent document FI130159.

The measurement of material components of the web may relate to solid material components such as dry stuff content OD (oven dry) and to mass of water per unit area i.e. water weight WW and they can be measured based on the 15 absorption of infrared radiation.

Measuring the total mass per unit area of the web with beta-ray absorption is a viable solution. The current challenge is the availability of beta sources. On the other hand, a measuring device based on beta-ray absorption requires licenses, training and other measures related to radiation safety, which cause work and costs for the customer. Neither the beta-ray absorption measurement device nor an X-ray absorption measurement device can be used to measure the proportions of different material components.

Despite the challenges, a measuring device based on beta-ray absorption is used because, for example, ash content of the web disturbs the measurements of the dry stuff content performed merely using electromagnetic radiation i.e. when NIR and X-ray radiation is used. Recycling increases uncertainty of ash content of a machine stock because ash content of partial stocks fed to a paper or board machine is not known. That leads to unreliable measurement results and potential variation of the end product when the control is based on X-rays and NIR. Although the beta-ray measurement provides reliable information on the web, it cannot easily be utilized because of its radioactivity and consequential dangers to people and the environment. Hence, an improvement would be desirable.

### BRIEF DESCRIPTION

The present invention seeks to provide an improvement in the measurements.

The invention is defined by the independent claims. Embodiments are defined in the dependent claims.

If one or more of the embodiments is considered not to fall under the scope of the independent claims, such an embodiment is or such embodiments are still useful for understanding features of the invention.

### LIST OF DRAWINGS

Example embodiments of the present invention are described below, by way of example only, with reference to the accompanying drawings, in which

FIG. 1A illustrates an example of optical and X-ray sensors performing transmission measurement;

FIG. 1B illustrates an example where the optical source and the optical detector of the optical sensor are on the same side of the web;

FIG. 2 illustrates an example of attenuation of optical radiation in interaction with the web of paper or board;

FIG. 3 illustrates an example of a paper machine;

FIG. 4 illustrates an example of a roll-to-roll process;

FIG. 5 illustrates an example of traversing measurement device;

FIG. 6 illustrates an example of a data processing unit;

FIG. 7 illustrates an example of variation of spectrum with different material components and example wavelengths for optical measurements; and

FIG. 8 illustrates of an example of a flow chart of a measuring and control method.

### DESCRIPTION OF EMBODIMENTS

The prior art measurement of total mass per unit area based on an Xray absorption can be used to determine paper, board and nonwoven products that 5 contain no minerals or are low in minerals. Variations in mineral content within the web 10 is a problem, because the response of X-rays with respect to minerals (e.g. carbonate and clay) is clearly higher than that to cellulose and water. In paper industry, recycled paper may have varying percentages of minerals leading to uncertainty in measurements based on X-rays.

Measuring various synthetic material components, binders or coatings with instruments based on fixed wavelengths requires the selection of optical filters for each application separately. If there are a lot of substances to measure, this is laborious and expensive. A spectrometer-based device does not have similar technical problems, but a spectrometer can be typically be used measure only the relative proportions of materials.

A spectrometer-based device does not have similar technical problems, but can be calibrated to show the proportion of each material component in the web 10. However, the challenge is that a spectrometer cannot typically measure the total mass per unit area or mass per unit area of each material component reliably. This is due to variations in the optical properties of the material being measured (e.g. scattering and changes in the response of the measured materials). Thus, a spectrometer can be typically be used measure only the relative proportions of materials in a reliable manner.

This document teaches a new measurement method to determine the total mass per unit area and mass per unit area of one or more material components of a moving web 10. The material components can be considered raw materials or structural component of the web 10. Note that the material components may include both solid material components and liquid material components, like water, for example.

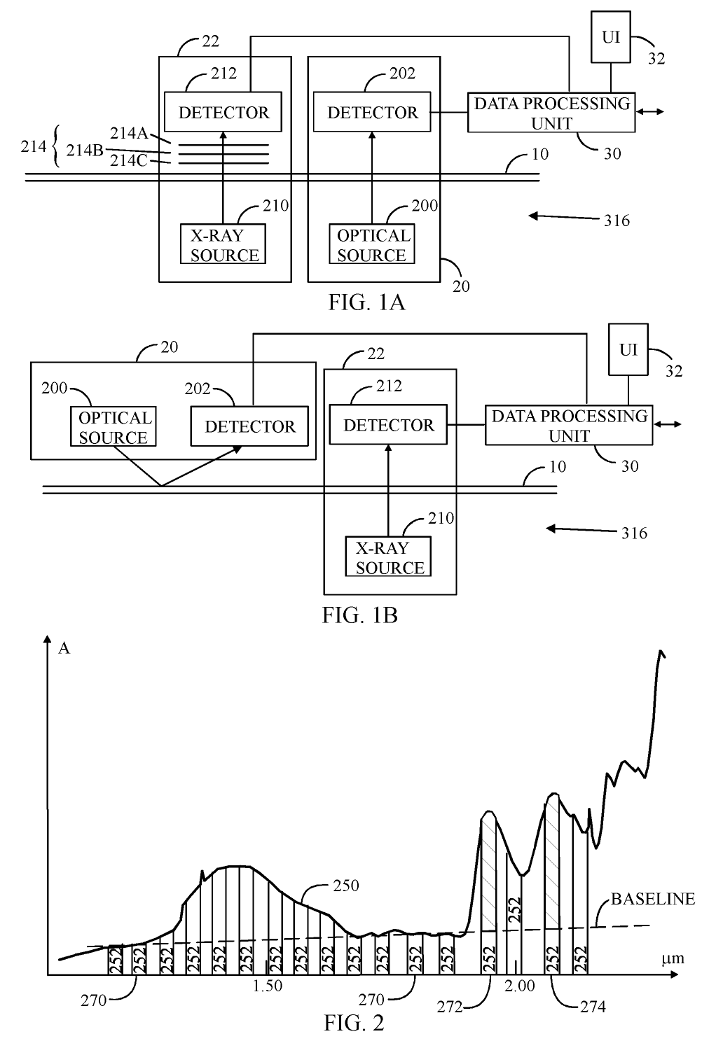

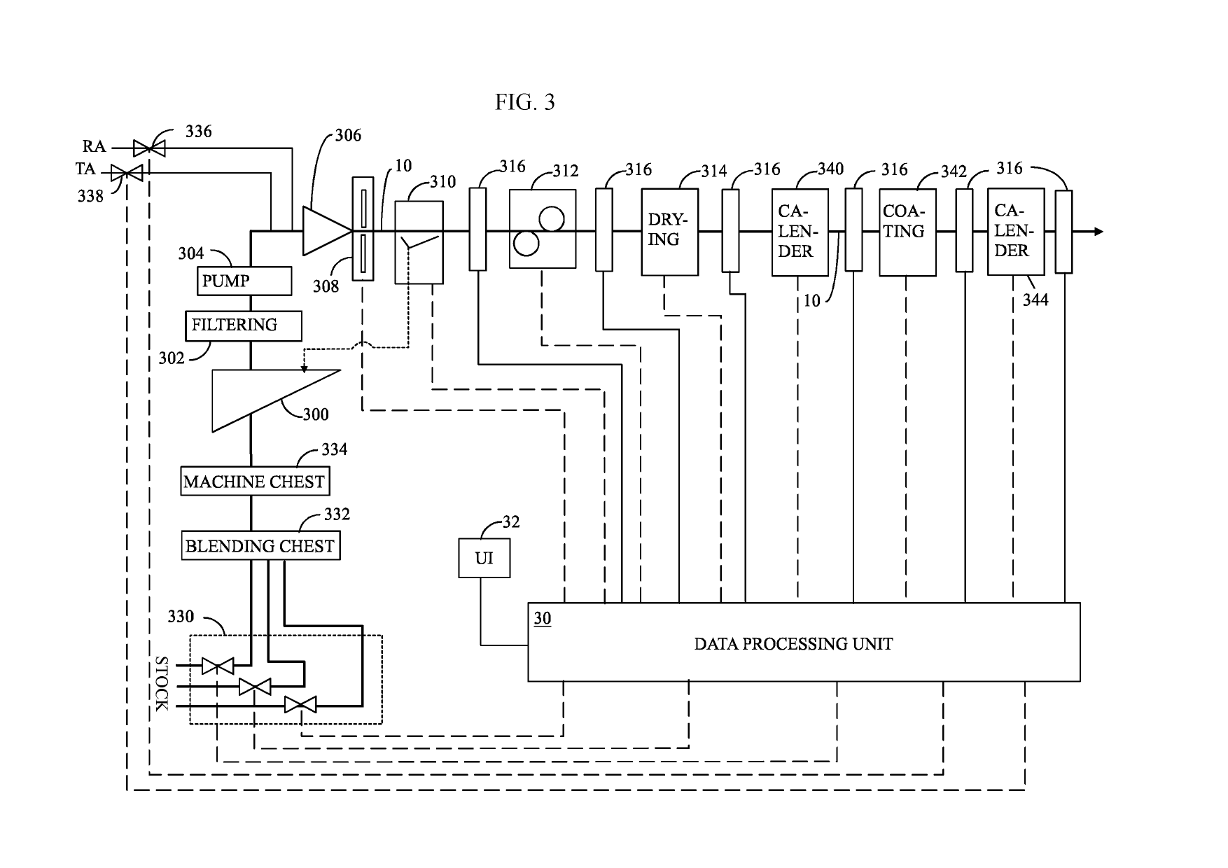

FIG. 1A illustrates an example of a measurement system for measuring a moving web 10. The measurement system comprises least one pair of sensors 20, 22 which are sensors 316 in FIG. 3 An X-ray sensor 22 and an optical sensor 20 of the pair sensors perform repeatedly electromagnetic transmission measurements of the web 10. The X-ray sensor 22 performs transmission measurements. The optical sensor 20 performs an optical measurement within an optical range, which includes characteristic optical absorption bands of material components of the web 10 and at least one reference band for the characteristic optical absorption bands. The optical sensor 20 may perform transmission measurements, which is illustrated in FIG. 1A, or backscattering measurements, see FIG. 1B. The material components that are wished to be measured may be called desired material components. That means, all or only a part of the material components of the web 10 may be measured.

The optical sensor 10 detects a plurality of optical bands 252 that are distributed over the optical range regularly or irregularly (the optical bands are shown in FIG. 2). The plurality of optical bands 252 may be mutually independent and they may be non-overlapping. The optical bands 252 are distributed in an independent manner with respect to both the one or more characteristic absorption bands of said at least one material components and the at least one reference band. No reference band has the same wavelength as any of the characteristic absorption bands. A number of the optical bands 252 of the optical range is equal to or larger than a combined number of the one or more characteristic optical absorption bands of the at least one desired material component and the one or more reference bands. The optical sensor 20 and the data processing unit 30 distinguish, in that manner, the one or more characteristic optical absorption bands of the at least one desired material component and the at least one reference band from each other.

The optical bands 252 can be considered sections or segments of the optical range. The optical bands 252 may be relatively narrow and they may correspond one-to-one with detector elements. In that manner, the number of the detector elements may define the number of the optical bands 252.

A data processing unit 30 receives data on repeated measurements. Then the data processing unit 30 fuses measurement data of the X-ray sensor 22 and the optical sensor 20 together, and forms information on at least one of the following based on the fused data: basis weight of the web 10 and a weight per unit area of at least one of the desired material components of the web 10. That the data processing unit 30 fuses the two data together can be considered such that the measurements although they are not directed to overlapping areas of the web 30 are assumed to be relate to the same measurement area (see FIG. 5).

Data fusion refers to integration of data from the optical sensor 20 and the X-ray sensor 22. Then, the data is merged and/or combined together. In that manner, it is possible to produce reliable, uniform, accurate and versatile information on the web 10. The information thus provided is typically more consistent than that provided by either of the X-ray sensor 22 or the optical sensor 20.

A person skilled in art is familiar with methods of combining measurement data of a plurality of sensors, per se.

The optical sensor 20 and the X-ray sensor 22 perform repeatedly electromagnetic transmission measurements of the web 20. The transmission measurement means that the electromagnetic radiation travels through the web 10.

The optical radiation may include infrared radiation in a NIR (Near InfraRed) region having a wavelength range required to achieve the measurement results. The wavelength range may cover about 750 nm to about 2500 nm and/or about 2500 nm to about 10 000 nm in a continuous or discrete manner. An optical source 200 of the optical sensor 20 directs the optical radiation to the web 10, and the optical radiation propagates through the web 10 to an optical detector 202. When the optical radiation propagates through the web 10, the optical radiation interacts with the web 10 and the light interacted with the web 10 carries information on the web 10 to the optical detector 202.

FIG. 1B illustrates an example where the optical source and the optical detector of the optical sensor are on the same side of the web. Also in this example, the optical radiation is output by the optical source 200, the optical radiation interacts with the web 10 and the optical radiation that has interacted with the web 10 is detected by the optical detector 202. In this example, it can be considered that the optical radiation reflects or backscatters from the web 10. It can be assumed the optical detector 202 may receive light that carries information on the whole thickness of the web 10 despite the optical source 200 and the optical detector 202 are on the same side of the web 10. The X-ray sensor 22 performs in this embodiment transmission measurements.

In an embodiment, the optical source 200 may output narrow measurement bands 252 that are detected by the detector (see FIG. 2). The optical source 200 may then comprise one or more leds (Light Emitting Diode), lasers or the like. Leds and lasers may output narrowband optical radiation at desired wavelengths.

In an embodiment, the optical source 200 may output broadband optical radiation which includes measurement bands. The optical source 200 may then comprise one or more incandescent lamps, gas-discharge lamps or the like. The incandescent lamps and gas-discharge lamps output broadband optical radiation that include more than one of the measured optical wavelengths.

Narrow measurement bands 350 may be formed although the optical source 200 is a broadband source. Namely, between the optical source 200 of broadband radiation and the optical detector 202 there may be one or more filters that band pass the measurement wavelength bands 350 for the detector 202 while fully or partly blocking other wavelengths. The filters may be attached with the optical source 200 and/or the optical detector 202. Alternatively, the filters may be separate from the optical source 200 and the optical detector 202.

The optical detector 202 detects the optical radiation that originates from the optical source 200 and that has been in interaction with the moving web 10. The interaction means that optical radiation passes through the web 10, whereby the intensity of optic radiation attenuates wavelength selectively depending on material components of the web 10.

FIG. 2 illustrates an example of absorption curve 250 of the web 10 and the optical measurement bands 252 that are distributed over the optical range of the measurement. The y-axis is attenuation or absorbance in an arbitrary scale. FIG. 2 relates to the web 10 of a paper and board industry but it is easy to generalize to any web 10 such as that of a nonwoven fabric. The optical measurement bands 252 may be distributed regularly or irregularly. In FIG. 2, which is a mere example, the optical measurement bands 252 are distributed almost regularly but as can be seen there are also slight irregularities for illustrating also them. That is, the distance between consecutive optical measurement bands 252 can be constant if the distribution is regular. If the distribution is irregular, the distance between consecutive optical measurement bands 252 is not constant. The distance can be defined to be measured between the middle points of each optical measurement band 252, for example. Additionally, different optical measurement bands 252 may have varying bandwidths instead of a constant bandwidth in an embodiment. The optical measurement bands 252 are distributed in an independent manner of both the characteristic absorption bands 272, 274 and the at least one reference band 270 of the material components. In that manner, the same measurement system can be used for various webs 10 that may have one or more different compositions of material components. A number of the optical bands 252 of the optical range of the measurement is equal to or larger than a combined number of the characteristic optical absorption bands of the desired material components and the reference bands.

In paper processing, optical bands 270, 272, 274 among all the measurement bands 252 may be utilized. The x-axis is wavelength in micrometers. Water has a characteristic absorption band 272 at about 1.95 μm (wavenumber about 5130 cm-1), for example, and thus moisture percentage or water mass per unit area of the web 10 may be measured at the band 272. Cellulose, which is a material component of the web 10, has a characteristic absorption band 306 at about 2.11 μm (wavenumber about 4740 cmn¹), for example, and thus cellulose mass per unit area of the web 10 may be measured at the band 274. Characteristic absorption of the optical radiation takes place at a wavelength where a local maximum of absorption is. Peaks 272, 274 of absorption are local maximums soaring above their environment.

Reference measurements to determine the relative water absorption and the relative cellulose absorption should be measured at wavelength bands where neither water nor cellulose has characteristic absorption. Such non-characteristic absorption wavelength bands i.e. reference bands 270 may be at about 1.8 μm (wavenumber about 5555 cmn¹) and about 1.3 μm (wavenumber about 7692 cm-1), for example, without limiting to these. A baseline, which the person skilled in the art is, per se, familiar with, may also be determined with the reference measurements. The baseline defines a general tilt of the attenuation as a function of the wavelength. The hatched area above the baseline at the characteristic wavelengths represent strength of attenuation or absorption.

Talc may be measured at about 1390nm, clay may be measured at about 2208nm and calcium carbonate may be measured at about 3980nm, for example.

The technical solution described in this document can be applied in paper and board manufacturing processes and in a manufacturing process of non-woven fabric.

Examine first an example of a paper machine illustrated in FIG. 3. One or more stocks, which may include ash, cellulose and/or water as material components of the web 10, are fed onto a paper machine through a wire pit silo 300, which is usually preceded by a blending chest 332 for partial stocks and a machine chest 334. The machine stock is dispensed for a short circulation, for instance, controlled by a basis weight control or a grade change program. The blending chest 332 and the machine chest 334 may also be replaced by a separate mixing reactor (not shown in FIG. 3), and the dispensing of the machine stock is controlled by feeding each partial stock separately by means of valves or another flow control means 330. In the wire pit silo 300, water is mixed into the machine stock to obtain a desired consistency for the short circulation (dashed line from a former 310 to the wire pit silo 300).

From the obtained stock it is possible to remove sand or the like (centrifugal cleaners), air (deculator) and other coarse material (pressure filter) using cleaning devices 302, and the stock is pumped with a pump 304 to a headbox 306. The sand or the like that avoids removal may form a part of ash in paper or board. Before the headbox 306, it is possible to add to the stock, in a desired manner, a filler TA, which includes one or more material components of the web 10, including e.g. gypsum, clay, calcium carbonate, talc, chalk, titanium dioxide and diatomite etc. and/or a retention agent RA, which also includes one or more material components of the web 10, such as inorganic, inartificial organic or synthetic water-soluble organic polymers. The filler TA and/or the retention agent RA may include ash as a material component ofthe web 10. A person skilled in the art is familiar with fillers and retention agents, per se.

Ash is what remains of paper or board after combustion at 900°C according to TAPPI T 413 "Ash in Wood, Pulp, Paper and Paperboard: Combustion at 900°C". According to TAPPI T 211 om-02, igniting papers or pulp containing no added fillers or coatings at either 525°C or 900°C will yield essentially identical results of a few tenths percent ash or less.

According to the standard (TAPPI T 211 om-02), ash may include various residues from chemicals used in paper or board manufacture, metallic matter from piping and machinery, mineral matter in the pulp from which the paper was made, and/or filling, coating, pigmenting and/or other added materials. In general, ash is material of paper or board that does not combust. All these substances can be considered material components of the web 10.

From the headbox 306 the stock is fed through a slice opening 308 of the headbox to a former 310, which may be a fourdrinier wire or a gap former. In the former 310, water drains out of the web 10 and additionally ash, fines and fibres are led to the short circulation. In the former 310, the stock is fed as a web 10 onto a wire, and the web 10 is preliminary dried and pressed in a press 312. The web 10 is actually dried in driers 314. In general, there is at least one pair of sensors 316 that performs an optical measurement and an X-ray measurement for determining basis weight and/or dry stuff content of the web 10. The at least one pair of sensors 316 feeds the measured data to a data processing unit 328 (see continuous lines therebetween). The basis weight may be considered the same as the dry stuff content if water weight is not taken into account. Additionally, the basis weight may be called grammage.

The paper or board machine may also include a pre-calender 340, a coating section 342 and/or a finishing calender 344. It is not necessary to have the coating section 342, however, and therefore it is not necessary to have more calenders 340, 344 than one. In the coating section 342, coating paste, which includes one or more material components may include ash, may be spread onto paper. The coating paste may include gypsum, clay, talc or carbonate, starch, latex or the like as material components of the web 10, for example. A person skilled in the art is familiar with the coating paste and its substances, per se.

In calenders 340, 344, where the uncoated or coated paper or board web runs between the rolls pressing with desired force, it is possible to change the surface properties of the paper, such as smoothness, roughness, topography, gloss and the like. The calender 340, 344 may also affect the paper thickness and/or the basis weight or other mass per unit area of the paper or board, for example. In the calender 340, 344, the properties of the paper web may be changed by means of web moistening, temperature and nip pressure between the rolls. In addition to this, it is clear that the operation of a paper machine is known, per se, to a person skilled in the art, and therefore, it need not be presented in greater detail in this context.

FIG. 3 also shows a control arrangement of the paper machine. Then the data processing unit 30 may also perform the data processing for the control of the paper or board machine. Factors affecting the quality and grade change include, inter alia, the number and mutual proportion of partial stocks, the amount of filler, the amount of retention agent, machine speed, the amount of white water and drying capacity. The data processing unit 30 may control the dispensing of partial stocks by means of valves 330, the dispensing of each filler TA by means ofthe valve 338, the dispensing of the retention agent RA by means of the valve 336, adjust the size of the slice opening 308, control the machine speed, control the amount of white water and the drying process in block 314 (see dashed lines). The data processing unit 30 utilizes the pair of the sensors 316 for measuring the web 10. The data processing unit 30 may also measure the properties of the web 10 elsewhere (e.g. at the same locations where controls are performed).

The data processing unit 30 may be conceived as a paper machine's control arrangement, or part thereof, based on automatic data processing. The data processing unit 30 may receive digital signals or convert the received analog signals to digital ones. The data processing unit 30 may comprise at least one processor and at least one memory and execute the signal processing in accordance with an appropriate computer program. The operating principle of the data processing unit 30 may be, for instance, PID (Proportional-Integral-Derivative) MPC (Model Predictive Control) or GPC (General Predictive Control) control.

In an embodiment when the web 10 is a non-woven fabric, the system performs the measurements in a similar manner to the measurements of the paper and board. The optical sensor 20 performs repeatedly transmission measurements through the moving web 10 at at least one wavelength band characteristically absorbed by the web 10 of the non-woven fabric. The X-ray sensor 22 performs repeatedly transmission measurements through the moving web 10 of the non- woven fabric. In that manner, the total mass per unit area of the web 10 can be measured. The data processing unit 30 receives data on the repeated measurements, fuses measurements of the X-ray sensor 22 and the optical sensor 20 together in a corresponding manner to the case where the web 10 is paper or board. Then the data processing unit 30 forms information on at least one of the following based on the fused data: basis weight of the web 10 of the non-woven fabric and a weight per unit area of at least one of the material components of the web 10 of the non-woven fabric.

Raw materials of non-woven fabric may include polymers, such as polypropylene, polyester, nylon, artificial or natural fibers, pulp and recycled fibers. The non-woven fabric may include in addition to the main material such as polymer or pulp one or more added material components. Here are examples of them. Adhesives and binders may be used to attach fibers of the non-woven fabric together and/or to adhere additional layers or materials to the non-woven fabric, which may strengthen the fabric and enhance durability. In some embodiments flame retardants are added to the non-woven fabric to make it fire resistant. Also antimicrobial agents may be added to some non-woven fabrics to make them resistant against microorganisms such as bacteria viruses and fungi, for example. Absorbents may be added to improve the fabric's ability to retain liquids. In some applications antistatic agents that are added to non-woven fabrics in order reduce static electricity and its build-up. Ultraviolet radiation stabilizers may be added to improve tolerance to UV light (for outdoor use). Softening agents may be added to make the non-woven fabric soft and comfortable. Colorants and dyes may be added to color the fabric. Reinforcing fibers such as glass or carbon fibers may be incorporated to bring strength and durability to the non-woven fabric. Moisture barrier coatings may be added to increase resistance to water and/or other liquids. All these examples of added materials are material components in the web 10 of the non-woven fabric, and their relative amount a mass per unit area in the web 10 may be measured using the optical sensor 20.

The measurement system for measuring the web 10 that may be paper, board or non-woven fabric can be implemented as a combination of the X-ray sensor 22 and an optical sensor 20 that performs measurement of the web 10 in NIR. The X-ray sensor 22 may considered a low-energy X-ray sensor. The data they produce is used in such a way that the total mass per unit area of the web 10, the mass per unit area of one or more material components of the web 10 can be measured. Potentially other web 10 characteristics may also be measured. The advantage of the low-energy X-ray measurement is that it does not require licences, training or other measures related to radiation safety.

The web 10 of paper, board and/or non-woven fabric may be a multilayer web 10. A multilayer web 10 is a fabric structure composed of multiple layers of woven or non-woven materials stacked on top of each other. The layers of the multilayer web 10 may be connected together by yarns or some bonding techniques, for example.

The goal is a simultaneous measurement of total mass per unit area of the web 10 and the mass per unit area of one or more material components included in the web 10 can be achieved in several ways. In an embodiment, the mass per unit area of all material components is individually measured. A few examples are described below.

In an embodiment, the optical detector 202 may comprise one or more detecting elements. The detector 202 may comprise detecting elements in line or matrix formation. Then each optical measurement band 252 can be detected separately. In the corresponding manner, the X-ray detector 212 may have one or more detecting elements. The optical detector 202 may detect different wavelengths based on a spatial distribution of the wavelengths to different detector elements, a scan over the wavelength range by one or more detector elements and/or a Fourier transform infrared wavelength detections, which may have a Michelson interferometer that modulates the wavelengths for forming an interferogram. The data processing unit 30 may then perform a Fourier transform to convert the interferogram into information on the detected wavelengths.

The optical sensor 20 performs an optical measurement within an optical range that is considered cover at least partially the need of a quality control of the web 10. The optical range includes characteristic optical absorption bands of desired material components of the web 10 and at least one reference band for the characteristic optical absorption bands.

A data processing unit 30 of the measurement system receives data on the repeated measurements, integrates measurements of the X-ray sensor 22 and the optical sensor 20 together, and form information on at least one of the following: basis weight of the web 10 and a weight per unit area of at least one of the desired material components of the web 10.

In an embodiment, it is possible to use the measurement data output by the X-ray sensor 22 to fine-tune the data from the optical sensor 20 when fusing the data together. That is, the measurement based of the optical sensor 20 can be calibrated to match with the measurements of the X-rays sensor 22. The calibration information is stored in the data processing unit 30 for performing measurements of the web 10. The calibration may be done as follows.

The X-ray measurement performed by the X-ray sensor 22 may be calibrated to show the total mass per unit area of the web 10 using laboratory references of the web 10. The laboratory references have predetermined total masses per unit area. The measurements of the optical sensor 20 are pre-calibrated to show masses per unit area of different material components in the web 10. Sufficient amount of data is collected at the production line for the calibration, where the mass per unit area and the relative share of material components varies in a known manner. The term sufficient depends on how accurate the measurement needs to be. What is sufficient may depend on standards of quality control, compliance, how to maximize production efficiency and/or how to minimize waste, for example. A sufficient amount of data typically covers data from the main products of the production line and the end-user requirements on product quality. The measurements of the optical sensor 20 may be calibrated such that the sum of the masses per unit area of the different material components is equal to the mass per unit area of the web 10 measured by the X-ray sensor 22. That may be done over the entire period of collected data. In addition, fine tuning can use other known features in the data that link the proportions of material components and the total mass per unit area, as well as their mutual variation.

An example of this kind of data is spectral libraries of various material components. A spectral library is a collection of spectral data that contains the unique spectral information on material components used in the process. The spectral library can be used as a reference. In that manner, identification and quantification of material components during calibration may be performed by matching their spectra to the data of known spectra of known material components stored in the spectral library.

An extended spectral library may take into account a response time of the measurement system, noise properties of the measurement system and environmental variables. Noise properties of the measurement system may mean characteristics of the unwanted disturbances or random variations that have effect on the end product and/or performance of the production system. The noise properties may include the strength of noise, the power of noise distributed over frequencies, temperature dependency of noise, phase noise and the range of frequencies over which noise is spread, for example.

In an alternative or additional embodiment, the measurement data of the optical sensor 20 may be used to compensate for the dependence of the measurements of the X-ray sensor 22 on the material component of the web 10. That is, the measurements of the X-ray sensor 22 can be calibrated based on the measurements of the optical sensor 20. That may be done in the following manner.

The data processing unit 30 may be pre-calibrated to show the total mass per unit area of the web 10 using laboratory references. However, this calibration may to some extent depend on the proportions of the material components in the web 10. Thus, the following may be additionally done. The measurements of the optical sensor 20 are in the calibration to show the relative proportions of different material components of the web 10. The relative proportions of different material components measured by the optical sensor 20 are used to correct the material dependence of the measurements of the X-ray sensor 22. In this case, the X-ray attenuation of different material components in the energy range used needs to be known. That can be separately measured or taken from the spectral library, for example. The result is a measurement of the total mass per unit area that is independent of the proportions of material components in the web 10 and a measure

In a third embodiment which may be used alone or with either or both of the previous embodiments. The data of the X-ray sensor 22 and the optical sensor 20 may be used together with the and physical, statistical and/or artificial intelligence-based models in calculations for forming the total mass per unit area and mass per unit area of the material components. Then, a model of data on the measurement of the X-ray sensor 22, the measurement of the optical sensor 20 and the properties of the web 10 is made based on physics, statistics and/or artificial intelligence, which predicts the results of the measurement of the X-ray sensor 22 and the measurement of the optical sensor 20 with a known variation of material components of web 10 properties (e.g. mass per unit area and material component ratios). The model is used to create an inverse model that predicts the total mass per unit area of a predetermined web 10 and masses per unit area of the predetermined material components of said web 10 based on the data produced by the X-ray sensor 22 and optical sensor 20. The inverse model derived from the predetermined web 10 is then used the total mass per unit area and masses per unit area of material components of a moving web 10 of the process based on data produced by the X-ray sensor 22 and the optical sensor 20.

In this document, the X-ray sensor is used to enable measurement of the one or more materials or components of the web 10. In one or more embodiments, the measurement is performed using electromagnetic radiation in a range about 1 keV to about 6 keV. In one or more embodiments, the measurement is performed using electromagnetic radiation in a range 1 keV to 5 keV. In one or more embodiments, the measurement is performed using electromagnetic radiation in a range 1 keV to 4 keV.

In an embodiment, the energy of the X-ray sensor 22 is limited to terminal energy of 5 kiloelectron volts. The advantage of using terminal energy of at or less than 5 kiloelectron volts is that no special radiation protection permits or training are required.

The X-ray measurement can be based on either single-channel measurement with suitable filters 214 that integrate the entire energy range used, or the X-ray measurement measuring several different energy bands with suitable filters 214A, 214B, 214C. The measurement of several X-ray channels can be carried out either with fixed filters, energy-dispersive solutions or wavelength dispersive solutions.

Examine now examples of the measurements in paper or board processes in more detail. The measurement apparatus comprises the optical sensor 20, which performs repeatedly transmission measurements through the moving web 10 of paper or board at at least one wavelength band characteristically absorbed by water, and at at least one wavelength band characteristically absorbed by cellulose. The apparatus also comprises the X-ray sensor 22, which performs repeatedly transmission measurements through the moving web 10 of paper or board with photons of electromagnetic radiation.

The apparatus further comprises the data processing unit 30, which receives signals with information on intensities of the optical and X-ray radiations passed through the web 10 from the optical sensor 20 and the X-ray sensor 22. The data processing unit 30 then determines, based on the information, all of the following of the web 10, the sheet moving in the machine direction: ash content, basis weight and dry stuff content. The information on the intensities may include attenuation or absorption information caused by the web 10 to the optical and X-ray radiation intensities, and/or percentages of the optical and X-ray radiation powers passed through the web 10.

A basis weight BW of the web 10 is a combination of the mass per unit area of all material components of the web 10. In paper and board industry basis weight is the weight of water WW and mass per unit area of dry stuff OD, i.e. BW = WW + OD. The dry stuff content may also be called an oven dry weight. The basis weight is an areal density of the web 10, which refers to its mass per unit of area. The basis weight may also be called grammage.

The data processing unit 30 can determine ash mass per unit area of the web 10 based on the transmission measurements with the photons of electromagnetic radiation. Then the data processing unit 30 may determine cellulose mass per unit area of the web 10 based on the optical transmission measurements at the at least one wavelength band 306 characteristically absorbed by cellulose. The data processing unit 30 can then determine the dry stuff content as a function of the cellulose mass per unit area of the web 10, the water weight per unit area and the ash mass per unit area of the web 10. The mass of ash per unit area may be formed based on the ash percentage or relative ash content that is measured with the X-ray measurement.

In an embodiment, the data processing unit 30 may determine water mass per unit area of the web 10 based on the transmission measurements at the at least one wavelength band 304 characteristically absorbed by water, and determine the basis weight as a function of the dry stuff content and the water mass per unit area.

Continue with the example relating to the paper and board process. In order to measure attenuation or absorption, intensity of the infrared radiation at a wavelength characteristically absorbed by water is measured and compared with intensity of the infrared light measured at a wavelength apart from a wavelength characteristically absorbed by water, cellulose and ash. Correspondingly, intensity of the infrared radiation at a wavelength characteristically absorbed by cellulose is measured and compared with intensity of the infrared light measured at a wavelength apart from a wavelength characteristically absorbed by water, cellulose and ash.

In order to calibrate the apparatus, attenuation or absorption of the infrared radiation originating directly from the optical and X-ray sources 200, 210 may be measured. Then any variation of the intensity of the optical and X-ray sources 200, 210 may be detected and compensated. The calibration may also include a measurement of a dark noise current of the detectors 202, 212. The calibration may be performed beside the web 10, where no measurement target is or where a known and/or predetermined reference target is.

In an embodiment, the data processing unit 30 may fuse the measurements of the web 10, which may be paper, board and/or non-woven fabric, performed by the X-ray sensor 22 and the optical sensor 20 together based on an iteration process. The data processing unit 30 may form percentages of the material component on the basis of the optical measurements. The data processing unit 30 may perform a conversion of the percentages into weights per unit area, and compare a sum the weights per unit area of the conversion with a total weight per unit area of the web 10 based on the X-ray measurement.

In an embodiment, the data processing unit 30 may fuse the measurements of the X-ray sensor 22 and the optical sensor 20 together based on the lookup-table process. Then the data processing unit 30 may form percentages of the material components on the basis of the measurements by the optical sensor 20, perform a conversion of the percentages into weights per unit area based on the lookup-table of absorption of the material components of the web 10, and compare a sum the weights per unit area of the conversion with a total mass per unit area of the web 10 based on the measurement by the X-ray sensor 22.

In an embodiment, the detector 202 may comprise a part that spreads different wavelengths of the broadband optical radiation into spectrum such that at least two detecting components detect different wavelength bands. The part spreading radiation into a spectrum may comprise a prism or a lattice. The spectrum indicates the intensity or attenuation of radiation as a function of the wavelength.

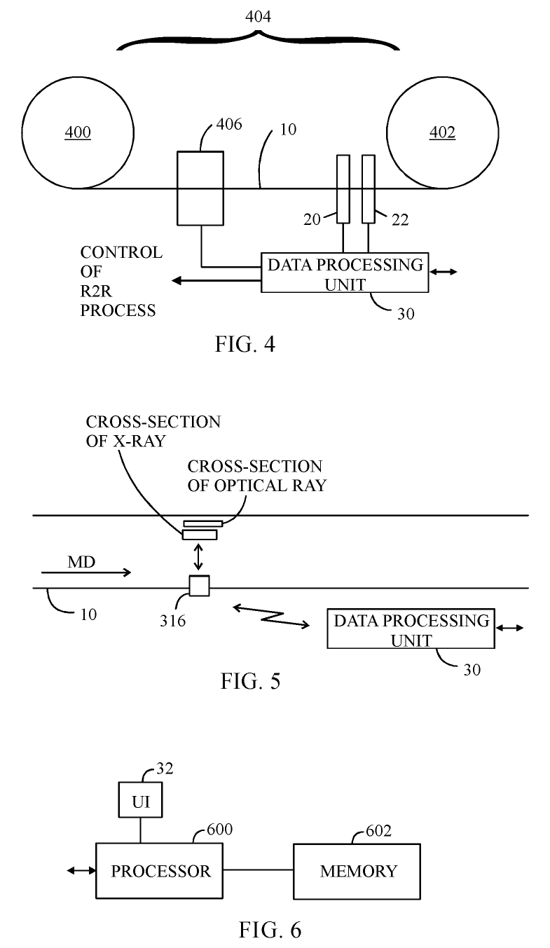

FIG. 4 illustrates an example of a roll-to-roll process, which may also be called a reel-to-reel process. The roll-to-roll process may have the moving web 10 of the non-woven fabric. The roll-to-roll process may alternatively the moving web 10 of paper or board. The web 10 is first rolled on the first roll 400 and it is fed from the first roll 400 through the web path to the second roll 402. In the web path 404 may include various operations like coating, printing, laminating, or embedding other material components by one or more operational equipment 406, for example. A person skilled in the art is familiar with such operations and equipment, per se. The web path 404 may have at least one optical sensor 20 and at least one X-ray sensor 22 located at one or more points of the process. In that manner, the measurements of the web 10 may be performed before and/or after one or more operations. The measurement may be performed after all necessary operations have been applied to the web 10. That means the final web 10 may be measured. The data processing unit 30 may control the operations performed to the web 10 in the web path 404 in the similar manner to that explained of the paper and board process. Additionally or alternatively, the data processing unit 30 may control the roll-to-roll process.

As illustrated in FIG. 5, the measurement apparatus comprises the sensor 316, which may comprise at least one of the optical sensor 20 and the X-ray sensor 22, traverses over the moving web 10 in a cross direction that is perpendicular to the machine direction. The traverse is a zig-zag line because when the web 10 moves in the machine direction and the at least one pair of sensors 316 travels in a perpendicular direction the line of measurement becomes zig-zag over the web 10. The communication between the traversing sensors 20, 22 and the data processing unit 30 may be wired or wireless which is also shown in FIG. 5.

In an embodiment, an overlapping length of an X-ray beam and an optical beam of the X-ray sensor 22 and the optical sensor 20, respectively, may be a few millimeters at the web 10, for example. The overlapping length may refer to a physical spot sizes and/or the computational sizes. In an embodiment, the X-ray beam and the optical beam do not need to be overlapping on or at the web 10. It is enough that the measurements are performed close enough each other. If the measurement areas are overlapping or are close enough, depends on measurement resolution of the web 10. For example, if the resolution is 5 cm, for example, the Xray measurement and the optical measurement may be separated from each other upto 2.5 cm. The sampling theorem may be used to define the theoretical maximum difference between the X-ray and the optical measurements. That also helps decide if both spots of these two electromagnetic radiations should be overlapping or not.

In an embodiment, the X-ray sensor 22 and the optical sensor 20 may perform the measurements synchronously at a common cross direction overlapping area on and inside the web 10. Then, the data of the X-ray measurement and the optical measurement are associated with the same transverse locations of the web 10 for forming the basis weight and the weight per unit area of at least one desired material component of the web 10.

In an embodiment, the system may perform repeatedly transmission measurements through the moving web 10 of paper or board with optical radiation that includes, because of the distribution, at least one wavelength band characteristically absorbed by at least one substance of the ash. The data processing unit 30 may then receive a signal with information on absorption of ash from the optical sensor 20, and determine the basis weight and/or the dry stuff content based also on the information from the optical sensor 20. The optical sensor 20 may be used to provide information on mass of ash per unit area of the web 10. If mass of ash per unit area measured with the X-ray sensor 22 and mass of ash per unit area measured with the optical sensor 20 deviate more than a predetermined threshold, operation of the sensor 22 may be checked and the measurement system may be recalibrated. The predetermined threshold may adjustable and its value may depend on quality and/or grade of the web 10, power of the optic source 200, sensitivity of the detector 202, any combination of these or the like, for example.

In an embodiment, the optical sensor 20 may perform repeatedly transmission measurements through the moving web 10 of paper or board at at least one wavelength band characteristically absorbed by at least one substance of the ash. The wavelength band is included in the distribution of wavelength bands spread over the optical range regularly or irregularly. Each of the components of ash may have their own specified characteristic absorption wavelength band. The component of ash may be a substance of the filler TA. However, pulp of wood includes also naturally components of ash. The data processing unit 30 may then receive a signal with information on absorption of ash from the optical sensor 20, and determine the basis weight and/or the dry stuff content based additionally on the information on ash from the optical sensor 20.

In an embodiment, the optical sensor 20 may perform at least one reference transmission measurement through the web 10 at at least one wavelength band apart from the at least one wavelength band characteristically absorbed by water, ash and cellulose. The data processing unit 30 may then determine the basis weight and/or the dry stuff content based on strengths of absorptions of the at least one reference transmission measurement and the transmission measurements of water and cellulose. As can be seen in FIG. 3, absorption may be defined as a difference between a level (dashed line) of the reference bands 270 and the value at a peak of the absorption at wavelengths 272, 274 of characteristic absorption of water and cellulose. The web 10 of non-woven fabric has also corresponding reference bands. As an example, polypropene of the non-woven fabric has characteristic absorption at about 1720 nm.

The distances between the sources 200, 210 and detectors 202, 212 may kept constant or their distances may be measured and any effect on the optical and X-ray measurements of distance variance may be compensated. Correspondingly, the distances between the sources 200, 210 and the sheet and/or the detectors 202, 212 and the web 10 may kept constant or their distances may be measured and any effect on the optical and X-ray measurements of distance variance may be compensated.

In an embodiment, the data processing unit 30 of a paper and/or board process may control a slice opening 108 of the headbox 106 in a cross direction. In this manner, amount of the stock on former 110 may be controlled in the cross direction. The amount of the stock, in turn, has effect on the water weight, the basis weight and/or the ash content of the web 10. In this manner, the water weight, the basis weight, the cellulose content and/or the ash content of the web 10 may be controlled, and the basis weight, the cellulose content and/or the ash content of the final product i.e. paper or board may be controlled. Additionally, filler percentage and/or retention agent percentage may be controlled.

In an embodiment, the data processing unit 30 may control feed of at least one of partial stock, feed of filler, feed of retention agent and removal of sand based on the ash mass per unit area of the web 10.

In an embodiment of the non-woven fabric process, dosing of raw materials, binder and/or the like may be controlled. In an embodiment of the non-woven fabric process, percentages of material components of coating, lamination, embedded materials and ink used in printing may be controlled.

The X-ray source 210 outputs X-rays that have energy between about 1 keV and about 5 keV. Low energy X-rays of the range about 1 keV to about 5 keV is often referred to as soft X-rays and considered not harmful like beta-radiation can be, for example.

In an embodiment, the X-ray source 210 may comprise an X-ray tube, which is a well-known generator of X-rays. The X-ray tube is harmless when it does not have operational voltage switched on. There is no half-life of an isotope, and the X-ray tube is stable.

In an embodiment, the X-ray source 210 may comprise a radioactive isotope iron-55 (nnFe), for example. The emitted X-rays from the iron-55 material of the X-ray source 210 are almost monochromatic, and a constant intensity of the X-rays continues for years. The energy of photons of the X-rays from the iron-55 is about 6 keV.

In an embodiment, the X-ray source 210 may comprise an X-ray fluorescence (XRF) source, which emits fluorescent X-rays as secondary radiation caused by exciting source material with higher energy X-rays. These are only examples of the X-ray source 210. X-ray sources are well-known, per se, and a person skilled in the art is familiar with X-ray sources, per se.

In an embodiment, the X-rays detector 212 may comprise a semiconductor detector, for example. The X-ray detector 212 may comprise PIN diode (standard Si PIN diode, for example), silicon lithium (Si(Li)) detector, Ge(Li), Silicon Drift Detector SDD. The semiconductor detectors may require temperature information for calibration in order to increase accuracy of the measurement. In an embodiment, the X-ray detector 212 may comprise an ionization chamber which outputs an electric current proportional to the X-ray dose at each moment. The ionization chamber may require temperature and pressure information for increasing the accuracy of dose measurement. X-ray detectors are well-known, per se, and a person skilled in the art is familiar with X-ray detectors, per se.

The Si(Li) detector may detect X-rays in a range about 1.5 keV to about 5 keV. The Ge(Li) detector may detect X-rays at or below 5 keV. The lithium drifted silicon detector may be used for X-rays in a range about 4 keV to about 5 keV, for example.

In an embodiment an example of which is illustrated in FIG. 1, the X-ray sensor 22 may output and receive photons of electromagnetic radiation at an energy level 6 keV or higher. The X-ray sensor 22 may comprise a first low pass filter 214A for a measurement of X-ray radiation energy in a first band equal to and smaller than 4 keV. In an embodiment, the measurement system may comprise a second low pass filter 214B for a measurement for X-ray radiation energy in a second band equal to and smaller than 5 keV. The X-ray sensor 22 may perform the measurement without the first and the second filter in the third band, and the data processing unit 22 may form a total energy of each of the X-ray bands. The data processing unit 30 may then determine weight per unit area of titanium oxide, clay and calcium carbonate of the web 10 based on relative absorptions of the three measurement bands, titanium oxide, clay and calcium carbonate being ash components of the web 10. Because their X-ray absorptions differ from each other, it is possible to easily compute their percentages in the web 10.

In an embodiment, the X-ray sensor 22 comprises a third low pass filter 214C for a measurement for X-ray radiation energy equal to and smaller than 6 keV. The X-ray sensor 22 may then perform a measurement with the third low pass filter in the third band.

In an embodiment, the X-ray sensor may comprise an X-ray spectrometer providing measurements that the data processing unit 30 may use for determining at least one material component even without the optical measurements. The data processing unit 30 may then define and control the basis weight of the web 30 directly.

FIG. 6 illustrates an example the data processing unit 30, which may comprise one or more processors 600 and one or more memories 602 including computer program code. The one or more memories 602 and the computer program code may, with the one or more processors 600, cause the measurement system to receive data on the repeated measurements, fuse data from the X-ray sensor 22 and the optical sensor 20 together, and form information on at least one of the following based on said fused data on absorption: basis weight of the web 10 and a weight per unit area of at least one of the at least one desired material component of the web 10.

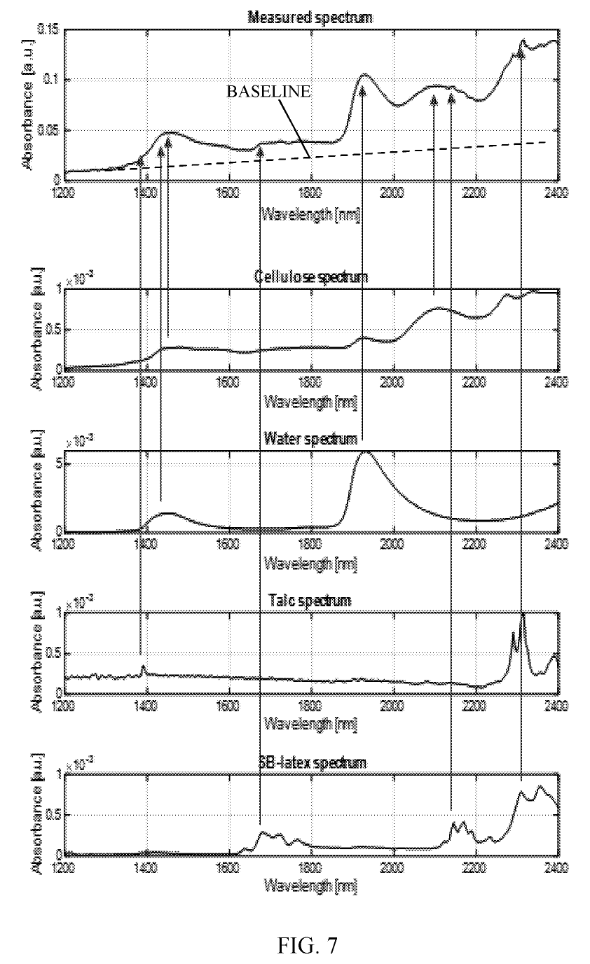

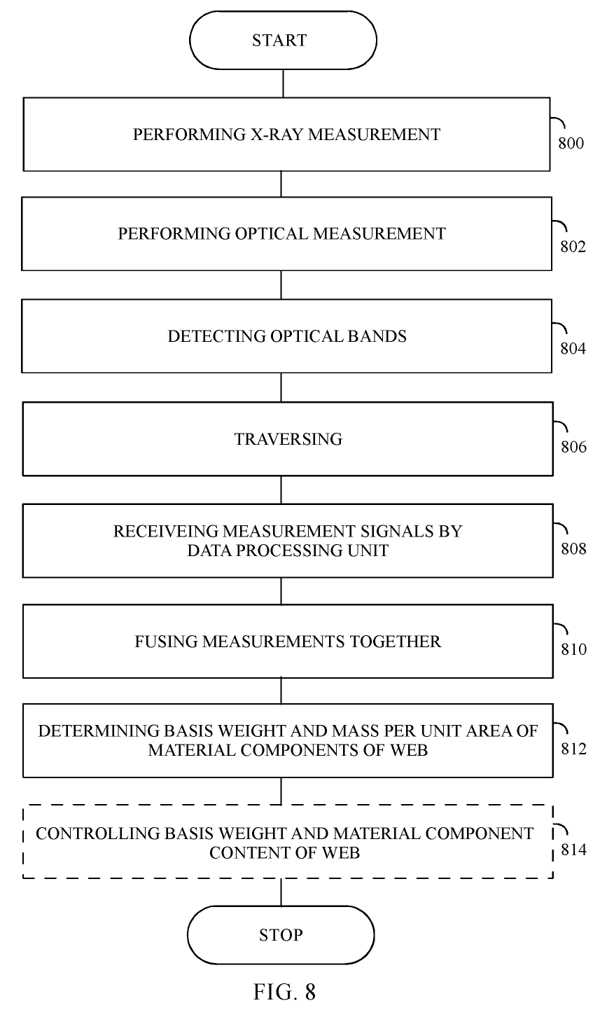

FIG. 7 illustrates an example of variation of absorbance with different material components of the web 10 and example wavelengths for optical measurement. Examples of characteristic absorption bands relate to talc, water, cellulose and SB-latex (Styrene-Butadiene latex). FIG. 7 also shows how different material component affect the overall absorption that is presented at the top of FIG. 7. A person skilled in the art is familiar with characteristic absorption bands of material components that are desired to be measured. A person skilled in the art is also familiar with material components that are used in various webs 10 and their optical features.

In an embodiment, a baseline curvature of the optical range is formed, and the basis weight based on absorptions of the infrared light of the detected 30 elements relating to the material components is determined. Then the basis weight and the weight per unit area of the at least one of the desired material components are corrected based on the baseline curvature. The tilt has an effect on the absorption values because the peak of the absorption may be too high without the correction. That can be seen in FIGS. 2 and 7. Namely, if the baseline were a horizontal line, i.e. the slope were 1, the peaks of characteristic absorption close to 2 μm would appear higher than with the correction of the baseline with a positive slope as a function of the wavelength that is shown with a dashed line in FIG. 2. Note also that with different reference measurements, the baseline may also have a negative slope. Then some peaks may appear lower than they actually are. That is why the peaks may be measured with respect to the estimated baseline at that wavelength.

FIG. 8 illustrates an example of a flow chart of the measurement and control method. In step 800, electromagnetic transmission measurements of the moving web 10 are performed repeatedly by an X-ray sensor 22 and an optical sensor 20.

In step 802, optical absorption measurements of the web 10 are performed within an optical range by the optical sensor 20, the optical range includes one or more characteristic optical absorption bands of at least one desired material component of the web 10 and at least one reference band for the characteristic optical absorption bands.

In step 804, which gives details how optical absorption measurements are performed, an optical measurement is performed within an optical range, which includes one or more characteristic optical absorption bands of at least one desired material components of the web 10 and at least one reference band for the optical absorption bands by the optical sensor 20. The optical measurement includes detecting, by the optical sensor 20, a plurality of optical bands 252 that are distributed over the optical range regularly or irregularly in an independent manner of both the one or more characteristic absorption bands and the at least one reference band of said desired material components, a number of the optical bands 252 of the optical range being equal to or larger than a combined number of the one or more characteristic optical absorption bands of the at least one desired material component and the reference bands for enabling the optical sensor 22 to distinguish the one or more characteristic optical absorption bands of the at least one desired material component and the at least one reference band from each other.

In step 806, at least one of the X-ray sensor (22) and the optical sensor (20) traverse across the moving web (10) in a transverse direction of the web (10).

In step 808, data on the repeated measurements is received by a data processing unit 30.

In step 810, measurements of the X-ray sensor 22 and the optical sensor 20 are fused together by the data processing unit 30.

In step 812, information is formed on at least one of the following by the data processing unit 30: basis weight of the web 10 and a weight per unit area of at least one of the desired material components of the web 10.

In an embodiment of step 814, the basis weight and mass per unit area of the material components of the web 10 may be controlled by the controller 30 based on the information on the at least one of the following by the data processing unit 30: basis weight of the web 10 and a weight per unit area of at least one of the desired material components of the web 10. A person skilled in the art is familiar with control, per se, while the information on the basis weight and the material component content of the web 10 is available.

In an embodiment, the information on basis weight is based on the measurement of the X-ray sensor 10.

In an embodiment relating to paper and board processes, feed of at least one of partial stock, feed of filler, feed of retention agent and removal of sand is controlled by the data processing unit 30 based on the basis weight and/or a weight per unit area of at least one of the desired material components of the web 10.

The method shown in FIG. 8 may be implemented as a logic circuit solution or computer program. The computer program may be placed on a computer program distribution means for the distribution thereof. The computer program distribution means is readable by a data processing device, and it encodes the computer program commands, carries out the measurements and optionally controls the processes on the basis of the measurements.

The computer program may be distributed using a distribution medium which may be any medium readable by the controller. The medium may be a program storage medium, a memory, a software distribution package, or a compressed software package. In some cases, the distribution may be performed using at least one of the following: a near field communication signal, a short distance signal, and a telecommunications signal.

A user interface 32 means an input/output device and/or unit. Non-limiting examples of a user interface include a touch screen, other electronic display screen, keyboard, mouse, microphone, handheld electronic game controller, digital stylus, display screen, speaker, and/or projector for projecting a visual display.

The technical solution presented in this document may replace beta absorption measurement that is used to measure a total mass per unit area of a web. The technical solution presented in this document makes it possible to accurately measure both the total weight per unit area of the web and the masses of material components of the web. The technical solution presented in this document enables the use of a low-energy X-ray source for measuring mass per unit area, which eliminates the need for permits, training and special arrangements related to radioactive radiation protection and safety.

An invention differs from a known technique in particular in that it makes use of the characteristics of two or more different measurement techniques and a combination thereof. The combination of measurements utilises the properties of each measurement technique, physical, statistical and/or artificial intelligence models and application knowledge, making the whole better than the sum of its parts.

The method shown in FIG. 8 may be implemented as a logic circuit solution or computer program. The computer program may be placed on a computer program distribution means for the distribution thereof. The computer program distribution means is readable by a data processing device, and it encodes the computer program commands, carries out the measurements and optionally controls the processes on the basis of the measurements.

1. A measurement system for measuring a moving web, wherein the measurement system comprises an X-ray sensor configured to perform repeatedly electromagnetic transmission measurements of the web of nonwoven fabric and/or cellulose, water and one or more ash components; the optical sensor configured to perform repeatedly optical absorption measurements of the web within an optical range, which is configured to include one or more characteristic optical absorption bands of at least one desired material component of the web and at least one reference band for the characteristic optical absorption bands; and the optical sensor being configured to detect a plurality of optical bands that are distributed over the optical range regularly or irregularly in a manner independent of both the characteristic absorption bands and the at least one reference band of said at least one desired material component, the number of the optical bands of the optical range being equal to or larger than the combined number of the one or more characteristic optical absorption bands of the at least one desired material component and the at least one reference band, the optical sensor being, in that manner, configured to distinguish the one or more characteristic optical absorption bands of the at least one desired material component and the at least one reference band from each other; at least one of the X-ray sensor and the optical sensor is configured to traverse across the moving web in a transverse direction of the web; and a data processing unit configured to receive data on the repeated measurements, fuse data from the X-ray sensor and the optical sensor together, and form information on at least one of the following based on said fused data on absorption: basis weight of the web and a weight per unit area of at least one of the at least one desired material component of the web.

2. The system of claim 1, wherein the system is configured to measure the at least one desired material component of the web that comprises one or more solid material components in a fiber form; the optical range that is configured to include one or more characteristic optical absorption bands of the at least one solid material component of the web and at least one reference band for the characteristic optical absorption bands which the optical sensor is configured detect; and the data processing unit is configured to form basis weight, and a weight per unit area of the at least one solid material component of the web.

3. The system of claim 1, wherein the optical range that is configured to include one or more characteristic optical absorption bands of the water, cellulose and at least one or more ash components of the web and at least one reference band for the characteristic optical absorption bands which the optical sensor is configured detect; and the data processing unit is configured to form basis weight, water weight per unit area, cellulose per unit area and at least one weight per unit area of the one or more ash components of the web.

4. The system of claim 2, wherein the system is configured to measure the at least one material component that is solid and includes at least one of the following: retention agent material, polymer material, monomer material, filling material, coating material and pigmenting material.

5. The system of claim 3, wherein the system is configured to measure the at least one ash component that includes at least one of the following: talc, clay, gypsum, starch, latex, calcium carbonate, titanium oxide.

6. The system of claim 1, wherein the system is configured to measure fossil-based polymer fibers, bio-based polymer fibers and/or modified polymer fibers of the web.

7. The system of claim 1, wherein the X-ray sensor is configured to output and receive photons of electromagnetic radiation at or below 5 keV.

8. The system of claim 1, wherein the X-ray sensor comprises a first filter configured to restrict output of the photons of electromagnetic radiation at or below 4 keV, the X-ray sensor is configured to perform the measurements with and without the first filter, and the data processing unit is configured to estimate percentage of calcium carbonate of the web based on absorptions of the measurements with and without the first filter, calcium carbonate being one ash component of the web.

9. The system of claim 1, wherein the optical sensor being configured to perform the measurements in the wavelength range 750 nm to 10 000 nm.

10. The system of claim 1, wherein the data processing unit is configured to match measurements of the X-ray sensor and the optical sensor together based on an iteration process and/or a lookup-table of absorption of the material components of the web at each of the measurement areas.

11. A measurement system for measuring a moving web, wherein the measurement system comprises an X-ray sensor configured to perform repeatedly electromagnetic transmission measurements of the web of nonwoven fabric and/or cellulose, water and one or more ash components; the optical sensor being configured to perform repeatedly optical absorption measurements of the web within an optical range, which is configured to include one or more characteristic optical absorption bands of at least one desired material component of the web and at least one reference band for the characteristic optical absorption bands, and the optical sensor being configured to detect a plurality of optical bands that are distributed over the optical range regularly or irregularly in an independent manner of both the one or more characteristic absorption bands and the at least one reference band of said at least one desired material component, the number of the optical bands of the optical range being equal to or larger than the combined number of the one or more characteristic optical absorption bands of the at least one desired material component and the at least one reference band, the optical sensor being, in that manner, configured to distinguish the one or more characteristic optical absorption bands of the at least one desired material component and the at least one reference band from each other; at least one of the X-ray sensor and the optical sensor is configured to traverse across the moving web in a transverse direction of the web; and a data processing unit is configured to receive data on the repeated measurements, fuse the data from the X-ray sensor and the optical sensor together, and form information on at least one of the following based on said fused data on absorption: basis weight of the web and a weight per unit area of at least one of the desired material components of the web, where the information on basis weight is based on the measurement of the X-ray sensor.

12. A measurement method of measuring a moving web, the method comprising performing, by an X-ray sensor, repeatedly electromagnetic transmission measurements of the moving web of nonwoven fabric and/or cellulose, water and one or more ash components; performing, by the optical sensor, optical absorption measurements of the web within an optical range, which includes one or more characteristic optical absorption bands of at least one desired material component of the web and at least one reference band for the characteristic optical absorption bands; detecting, by the optical sensor, a plurality of optical bands that are distributed over the optical range regularly or irregularly in an independent manner of both the one or more characteristic absorption bands and the at least one reference band of said desired material components, the number of the optical bands of the optical range being equal to or larger than the combined number of the one or more characteristic optical absorption bands of the at least one desired material component and the reference bands for enabling the optical sensor to distinguish the one or more characteristic optical absorption bands of the at least one desired material component and the at least one reference band from each other; performing transversing, by at least one of the X-ray sensor and the optical sensor, across the moving web in a transverse direction of the web; receiving, by a data processing unit, data on the repeated measurements; fusing, by the data processing unit, the data from the X-ray sensor and the optical sensor together; and forming, by the data processing unit, information on at least one of the following based on the fused data on absorption: basis weight of the web and a weight per unit area of at least one of the desired material components of the web.

13. The method of claim 12, the method further comprising performing a measurement of a multilayer web.

14. A measurement method of measuring a moving web, the method comprising performing, by an X-ray sensor, repeatedly electromagnetic transmission measurements of the moving web of nonwoven fabric and/or cellulose, water and one or more ash components; performing, by the optical sensor, optical absorption measurements of the web within an optical range, which includes one or more characteristic optical absorption bands of at least one desired material components of the web and at least one reference band for the characteristic optical absorption bands; detecting, by the optical sensor, a plurality of optical bands that are distributed over the optical range regularly or irregularly in an independent manner of both the one or more characteristic absorption bands and the at least one reference band of said desired material components, the number of the optical bands of the optical range being equal to or larger than the combined number of the one or more characteristic optical absorption bands of the at least one desired material component and the reference bands for enabling the optical sensor to distinguish the one or more characteristic optical absorption bands of the at least one desired material component and the at least one reference band from each other; transversing, by at least one of the X-ray sensor and the optical sensor, across the moving web in a transverse direction of the web; receiving, by a data processing unit, data on the repeated measurements; fusing, by the data processing unit, the data from the X-ray sensor and the optical sensor together; and forming, by the data processing unit, information on at least one of the following based on said fused data on absorption: basis weight of the web and a weight per unit area of at least one of the desired material components of the web, where the information on basis weight is based on the measurement of the X-ray sensor.

15. The method of claim 13, the method further comprising performing a measurement of a multilayer web.
